(19) United States
(12) Patent Application Publication
(10) Pub. No.: US 2005/0004364 A1
(43) Pub. Date: Jan. 6, 2005

(54) Process for the preparation and purification of 1,5-naphthyridine-3-carboxyamides and purification of 1,5-naphthyridine-3-carboxyamides

(75) Inventors: Michael J. Castaldi, Pawcatuck, CT (US); David H. B. Ripin, Westbrook, CT (US); Dennis E. Bourassa, Norwich, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/852,942

(22) Filed: May 25, 2004

Related U.S. Application Data
(60) Provisional application No. 60/473,823, filed on May 27, 2003.

(51) Int. Cl. C07D 471/02

(52) U.S. Cl. 544/331; 546/123

(57) ABSTRACT

A new route for the preparation and purification of substituted 1,5-naphthyridine-3-carboxyamides, useful in the diagnosis and treatment of anxiety, Downs Syndrome, sleep, cognitive and seizure disorders, and overdose with benzodiazepine drugs and for enhancement of alertness, is provided. These compounds may be readily prepared by treating the corresponding 1,5-naphthyridine-3-carboxylic acids with a primary amine and a 1,1-carbonyldiimidazole. Purification is achieved by converting the substituted 1,5-naphthyridine-3-carboxyamides to a salt with a strong base such as potassium t-butoxide, recrystallizing and acidifying to regenerate the pure carboxyamide.

### BACKGROUND OF THE INVENTION

This invention relates to a new route for the preparation and purification of substituted 1,5-naphthyridine-3-carboxyamides and the pharmaceutically acceptable non-toxic salts thereof. These compounds are highly selective agonists, antagonists or inverse agonists for GABAa brain receptors or prodrugs of agonists, antagonists or inverse agonists for GABAa brain receptors. These compounds are useful in the diagnosis and treatment of anxiety, Down Syndrome, sleep, cognitive and seizure disorders, and overdose with benzodiazepine drugs and for enhancement of alertness.

The substituted 1,5-naphthyridine-3-carboxyamides that are prepared in accord with the process of the present invention are disclosed in U.S. Pat. No. 6,143,760 and PCT International Publication No. WO99/10347 A1, each of which is incorporated herein by reference in its entirety.

### SUMMARY OF THE INVENTION

The present invention comprises a process of preparing a compound of the formula IV: 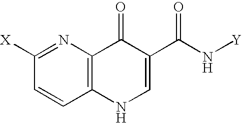
wherein X is hydrogen, halogen, —OR₁, C₁-C₆ alkyl optionally substituted with up to three groups selected independently from halogen and hydroxy, or, —NR₂R₃; phenyl, naphthyl, 1-(5,6,7,8-tetrahydro)naphthyl or 4-(1,2-dihydro)indenyl, pyridinyl, pyrimidyl, isoquinolinyl, 1,2,3,4-tetrahydroisoquinolinyl, benzofuranyl, benzothienyl, each of which is optionally substituted with up to three groups selected from halogen, C₁-C₆ alkyl, C₁-C₄ alkoxy, C₁-C₆ alkylthio, hydroxy, amino, mono or di(C₁-C₆)alkylamino, cyano, nitro, trifluoromethyl or trifluoromethoxy; or, a carbocyclic group containing from 3-7 members, up to two of which members are optionally hetero atoms selected from oxygen and nitrogen, where the X carbocyclic group is optionally substituted with one or more groups selected from halogen, alkoxy, mono- or dialkylamino, sulfonamide, azacycloalkyl, cycloalkylthio, alkylthio, phenylthio, or a heterocyclic group;
Y is lower alkyl having 1-8 carbon atoms optionally substituted with up to two groups selected from halogen, alkoxy, mono- or dialkylamino, sulfonamide, azacycloalkyl, cycloalkylthio, alkylthio, phenylthio, a heterocyclic group, —OR₄, —NR₅R₆, SR₇, or aryl; or a carbocyclic group having from 3-7 members atoms, where up to three of which members are optionally hetero atoms selected from oxygen and nitrogen and where any member of the Y carbocyclic group is optionally substituted with halogen, —OR₄, —NR₅R₆, SR₇, aryl or a heterocyclic group;
R₁ and R₄ are independently hydrogen, lower alkyl having 1-6 carbon atoms, or cycloalkyl having 3-7 carbon atoms, where each alkyl may be optionally substituted with —OR₄, or —NR₅R₆;
R₂ and R₃ are independently hydrogen, lower alkyl optionally mono- or disubstituted with alkoxy, aryl, halogen, or mono- or di-lower alkyl; aryl or aryl(C₁-C₆)alkyl where each aryl is optionally substituted with up to three groups selected from halogen, hydroxy, C₁-C₆ alkyl, C₁-C₆ alkoxy, or mono- or di (C₁-C₆) alkylamino; cycloalkyl having 3-7 carbon atoms optionally mono or disubstituted with halogen, alkoxy, or mono- or di-lower alkyl; or —SO₂R₈;
R₅ and R₆ have the same definitions as R₂ and R₃, respectively;
R₇ is hydrogen, lower alkyl having 1-6 carbon atoms, or cycloalkyl having 3-7 carbon atoms; and
R₈ is lower alkyl having 1-6 carbon atoms, cycloalkyl having 3-7 carbon atoms, or optionally substituted phenyl;
said process comprising the step of treating a compound of the formula III: 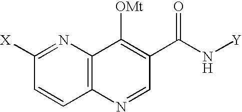
   wherein Mt is K, Na, Ce, Li or quaternary (C₁-C₆)alkyl ammonium, with a strong acid.

In a further aspect of the present invention, said compound of formula III, which is a salt of the compound of formula IV wherein X and Y are as defined above, is prepared by treating the compound of formula IV with a strong base, MtOBs, wherein Mt is K, Na, Ce, Li or quaternary alkylammonium, and Bs is H, or (C₁-C₆) alkyl, preferably potassium t-butoxide.

In another aspect of the present invention, said compound of formula III is purified by filtration and recrystallization from a solvent, preferably a mixture of a water miscible ethereal solvent, and more preferably a mixture of tetrahydrofuran and water.

In a further aspect of the present invention, said compound of formula IV is prepared by treating a compound of the formula II: 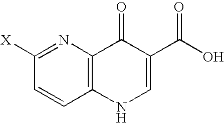
wherein X is as defined above, with a primary amine YNH₂ and a coupling agent, preferably 1,1-carbonyldiimidazole.

In another aspect of the present invention, said compound of formula II, wherein X is as defined above, is prepared by treating a compound of the formula I: 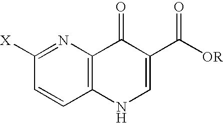
wherein R is C₁-C₆ alkyl, preferably ethyl, and X is as defined above, with (1) a strong base such as a hydroxide or an alkoxide base, preferably NaOH, KOH and KOC(CH₃)₃ and (2) an acid, preferably a strong acid.

In another aspect of the process of the present invention, in said acidification step (2), said compound of formula II, wherein X is as defined above, is prepared in a form whereby it may be readily and conveniently purified by filtration by adjusting the pH of the mixture resulting from treatment with base with a strong acid to about pH 1 to about pH 4, preferably about pH 2, aging at elevated temperature, cooling and filtering.

The compounds which are prepared by the process of the present invention can be described by general formulas I-IV set forth above. In a further embodiment of the process of the present invention, in any of the aforesaid general formulas I-IV, X or Y may be —NR₂R₃ which is a heterocyclic group such as, for example, piperidine in the case where R₂ and R₃ together form a C₅-alkylene group. Further, R₂ and R₃ together may represent an alkylene or alkenylene group optionally containing up to two heteroatoms selected from nitrogen and oxygen. The resulting groups include imidazolyl, pyrrolidinyl, morpholinyl, piperazinyl, and piperidinyl.

Similarly, the —NR₅R₆ group in formula I above can also represent a heterocyclic group such as, for example, piperidine in the case where R₅ and R₆ together form a C₅-alkylene group. Further, R₅ and R₆ together may represent an alkylene or alkenylene group optionally containing up to two heteroatoms selected from nitrogen and oxygen. The resulting groups include imidazolyl, pyrrolidinyl, morpholinyl, piperazinyl, and piperidinyl.

In certain embodiments of the invention, X represents (C₁-C₆) alkoxy, more particularly, (C₁-C₃)alkoxy. In certain specific embodiments, X is methoxy or ethoxy.

For the process of the present invention, other substituents for compounds of formulas III-IV include Y being lower alkyl, e.g., methyl or ethyl, optionally substituted with phenyl, pyridyl, or pyrimidinyl. In particular embodiments, the Y group is benzyl optionally substituted with halogen, (C₁-C₆)alkyl, (C₁-C₆)alkoxy, amino, or mono- or di(C₁-C₆) alkyl.

Where R₂ and R₃ in compounds I-IV represent optionally substituted aryl or aryl(C₁-C₆)alkyl, the aryl group may be phenyl, pyridyl, or pyrimidinyl, and the alkyl groups may be methyl or ethyl. Alternatively, R₂ and R₃ may be benzyl and phenyl.

Where X is optionally substituted C₁-C₆ alkyl, the alkyl group is optionally substituted methyl, ethyl, or propyl. Particular examples are perhalomethyl and trihaloethyl. Specifically, the halogens are fluorine. In a particular embodiment, substituted alkyl is 2,2,2-trifluoroethyl.

X in formulas I-IV may be an optionally substituted phenyl, naphthyl, 1-(5,6,7,8-tetrahydro)naphthyl, 4-(1,2-dihydro)indenyl, pyridinyl, pyrimidyl, isoquinolinyl, benzofuranyl, or benzothienyl group, or a 1,2,3,4-tetrahydroisoquinolinyl group.

In addition to the compounds of formulas I-IV, the process of the present invention encompasses preparation of compounds of formula IVA: 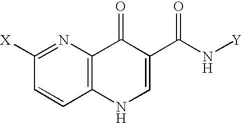
compounds of the formula IIIA: 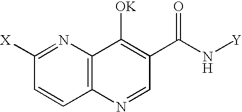
and compounds of the formula IIA: 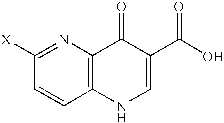
as well as compounds of the formula IA: 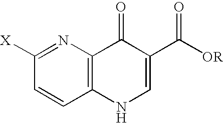
wherein substituent group X and substituent group Y when present either together or separately in any of the aforesaid general formulas IA, IIA, IIIA and IVA are defined as follows:
wherein X is:
(i) hydrogen, halogen, mono- or dialkylamino, alkoxy;
(ii) a group of the formula: 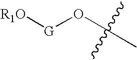
   where G is lower alkylene having 1-6 carbon atoms, or a cyclic group of the formula 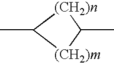
   where n is 0, 1, or 2, and m is an integer of from 1 to 5, with the proviso that the sum of n+m is not less than 1 or greater than 5; and R₁ is hydrogen, lower alkyl, or (C₃-C₇)cycloalkyl, where the alkyl or cycloalkyl is optionally substituted with halogen, lower alkoxy, or mono- or di(C₁-C₆)alkylamino;
(iii) a group of the formula: 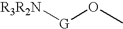
   where G is as defined above for ii; and R₂ and R₃ independently represent hydrogen, lower alkyl having 1-6 carbon atoms, cycloalkyl having 3-7 carbon atoms, —SO₂R₈ where R₈ is (C₁-C₆)alkyl, (C₃-C₇)cycloalkyl, or optionally substituted phenyl, or R₂ and R₃ together with the nitrogen atom to which they are attached form a heterocyclic moiety selected from imidazolyl, pyrrolidinyl, morpholinyl, piperazinyl, or piperidinyl;
(iv) a group of the formula: 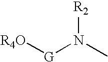
   where
R₂ is as defined above for iii;
R₄ is hydrogen, lower alkyl having 1-6 carbon atoms, or cycloalkyl having 3-7 carbon atoms, and may be optionally substituted with one or more (C₁-C₆)alkoxy or mono- or di(C₁-C₆)alkylamino groups; and
G is as defined above for ii;
(v) a group of the formula: 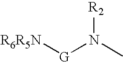
   where
R₂ and G are as defined above for iv and ii, respectively, and
R₅ and R₆ independently represent hydrogen, lower alkyl having 1-6 carbon atoms, cycloalkyl having 3-7 carbon atoms, —SO₂ R₈ where R₈ is (C-C₆)alkyl, (C₃-C₇)cycloalkyl, or optionally substituted phenyl, or
R₅ and R₆ together with the nitrogen atom to which they are attached form a heterocyclic moiety;
(vi) a group of the formula: 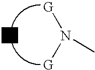
   where G is as defined above for ii; or
(vii) a group of the formula: 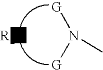
   where each G is as defined above for ii; and
Y is
(viii) lower alkyl having 1-8 carbon atoms or cycloalkyl having 3-7 carbon atoms, any of which may be optionally substituted with one or more hydroxy, halogen, (C₁-C₆)alkoxy, alkoxyalkoxy where each alkoxy is (C₁-C₆)alkoxy, (C₁-C₆) alkylthio, (C₃ -C₇)Cycloalkylthio, aryl, heteroaryl, or mono- or di(C₁-C₆)alkylamino groups;
(ix) a group of the formula: 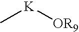
   where K is lower alkylene having 1-6 carbon atoms optionally substituted with (C₁-C₆)alkyl or alkylene, or a cyclic group of the formula 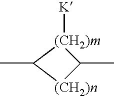
   where K′ independently represents hydrogen or (C₁-C₆) alkyl or alkylene, n is 0, 1, or 2, and m is an integer of from 1 to 5, with the proviso that the sum of n+m is not less than 1 or greater than 5; and
R₉ is hydrogen, lower alkyl, or (C₃-C₇)cycloalkyl, where the alkyl or cycloalkyl is optionally substituted with halogen, lower alkoxy, or mono- or dialkylamino;
(x) a group of the formula: 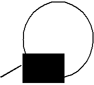
   where K is defined as above in ix;
(xi) a group of the formula: 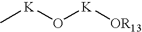
   where
K is as defined above for ix, and
R₁₃ is hydrogen, lower alkyl having 1-6 carbon atoms, or cycloalkyl having 3-7 carbon atoms, where the alkyl and cycloalkyl groups are optionally substituted with one or more (C₁-C₆)alkoxy or mono- or di(C₁-C₆)alkylamino groups; and
(xii) a group of the formula: 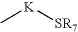
   where K is as defined above for ix, and
R₇ is hydrogen, lower alkyl having 1-6 carbon atoms, or cycloalkyl having 3-7 carbon atoms; and
(xiii) a group of the formula: 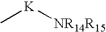
   where K is as defined above for ix; and
R₁₄ and R₁₅ independently represent hydrogen, lower alkyl having 1-6 carbon atoms, cycloalkyl having 3-7 carbon atoms, —SO₂ R₈ where R₈ is as defined above, or R₁₄ and R₁₃ together with the nitrogen atom to which they are attached form a heterocyclic moiety;
(xiv) a group of the formula: 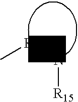
   where K and R₁₅ are as defined above in ix and xii, respectively;
(xv) a group of the formula: 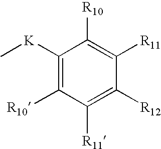
   where K is as defined above for ix;
R₁₀ and R_{10′} are the same or different and are selected from hydrogen, (C₁-C₆)alkyl, halogen, hydroxy, lower alkoxy having 1-6 carbon atoms, or cycloalkoxy having 3-7 carbon atoms;
R₁₁ and R_{11′}, and R₁₂ are the same or different and are selected from hydrogen, C₁-C₆ alkyl, halogen, hydroxy, —OR₄, —CR₇ (R₉)NR₅ R₆, —CR₉(R₁₆) OR₄, or R₁₁ and R₁₂ taken together with the atoms to which they are attached form a (hetero)cyclic ring; and
R₁₆ is hydrogen, lower alkyl having 1-6 carbon atoms, or cycloalkyl having 3-7 carbon atoms;
(xvi) a group of the formula: 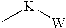
   where K is as defined above for ix; and W is heteroaryl;
(xvii) a group of the formula: 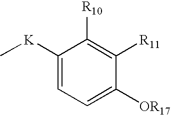
   where K is as defined above for ix; R₁₀ and R₁₁ are as defined above for xv, and R₁₇ is hydrogen, lower alkyl, or (C₃-C₇)cycloalkyl, where the alkyl or cycloalkyl is optionally substituted with halogen, lower alkoxy, or mono- or di(C₁-C₆)alkylamino;
(xviii) a group of the formula: 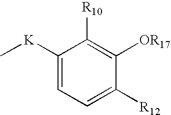
   where K, R₁₀, R₁₂, and R₁₇ are as defined above;
(xix) a group of the formula: 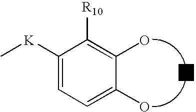
   where each K is independently as defined above for ix and R₁₀ is defined above
(xx) a group of the formula: 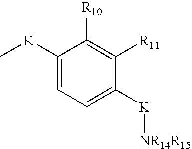
   where K, R₁₀, R₁₁, R₁₄, and R₁₅ are as defined above;
(xxi) a group of the formula: 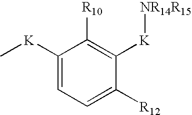
   where K, R₁₀, R₁₂, R₁₄, and R₁₅ are as defined above;
(xxii) pyrimidinyl(C₁-C₆)alkyl or pyridyl(C₁-C₆)alkyl; or
(xxiii) a group of the formula: 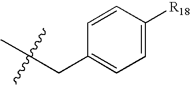
   where R₁₈ represents hydrogen, amino, mono-, or di(C₁-C₆)alkylamino, or C₁-C₆ alkyl optionally substituted with a R₁₉ where R₁₉ represents: 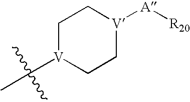
   where V and V′ are independently CH or nitrogen; A″ is C₁-C₆ alkylene; and
R₂₀ is phenyl, pyridyl, or pyrimidinyl, each of which is optionally mono-, di-, or trisubstituted independently with halogen, hydroxy, C₁-C₆ alkoxy, amino, or mono- or di(C₁-C₆)alkylamino.

Specific compounds made by the process of the invention include compounds wherein pyrimidinyl(C₁-C₆)alkyl Y groups are 2- and 4-pyrimidinylmethyl. In particular, pyridyl(C₁-C₆)alkyl Y groups are 2- and 4-pyridylmethyl.

Other compounds made by the process of the invention include compounds wherein benzyl Y groups are those where R₁₈ is amino or a substituted methyl or ethyl group. More particularly, R₁₈ substituents are piperazin-1-yl or piperidin-1-yl substituted at the 4-position with a halogenated benzyl group.

Other specific compounds made by the process of the invention include those wherein benzyl Y groups are 4-[1-[4-(4-fluorobenzyl)piperazinyl] methyl]benzyl and 4-[1-[4-(4-fluorobenzyl)piperidinyl]methyl]benzyl.

“X” groups in formula IVA are various quinolinyl, isoquinolinyl, tetrahydroquinolinyl, or tetrahydroisoquinolinyl groups, e.g., groups of the formulas:

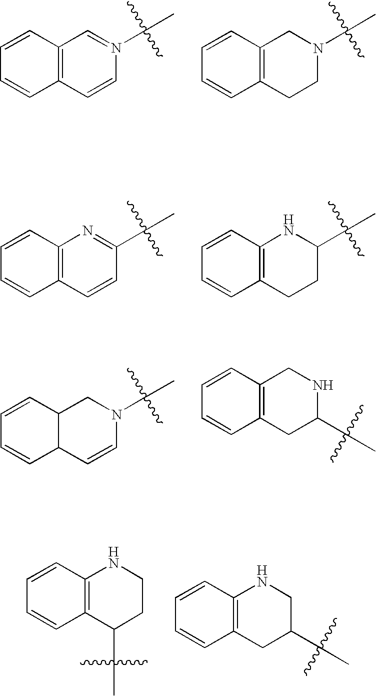

The following formulae represent embodiments that are prepared by the process of the present invention:
V: 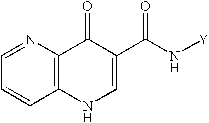
wherein Y is defined above.
VI: 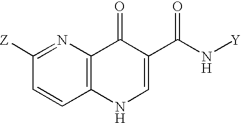
wherein Z represents halogen and Y is as defined above.
VII: 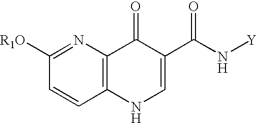
wherein R₁ and Y are defined above.
VIII: 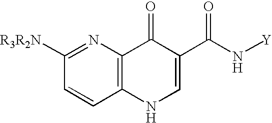
wherein R₂, R₃, and Y are defined above.
IX: 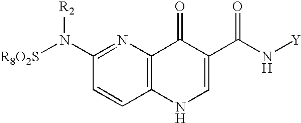
wherein R₂, R₈, and Y are defined above.
X: 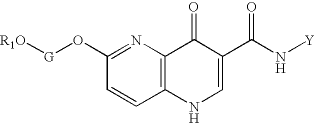
wherein R₁, G and Y are defined above.
XI: 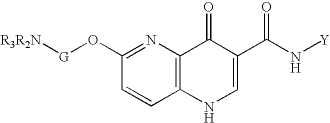
wherein R₂, R₃, G, and Y are defined above.
XII: 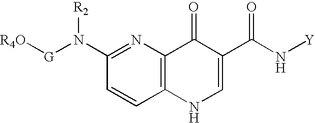
wherein R₂, R₄, G, and Y are defined above.
XIII: 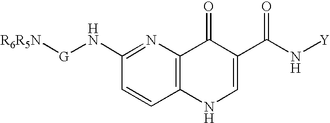
wherein R₂, R₅, G, and Y are defined above.
XIV: 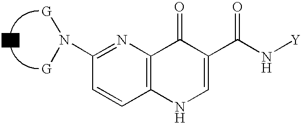
wherein G and Y are defined above.
XV: 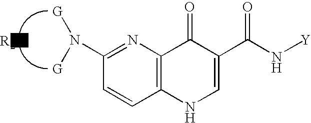
wherein R₂, G, and Y are defined above.
XVI: 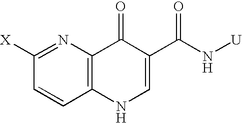
wherein X is defined above and U is (C₁-C₆)lower alkyl or (C₁-C₆) cycloalkyl.
XVII: 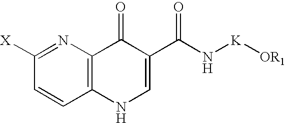
wherein X, K, and R₁ are defined above.
XVIII: 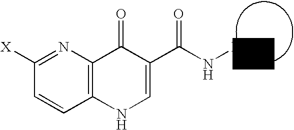
wherein X and K are defined above.
XIX: 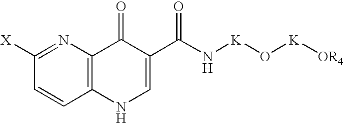
wherein X, K, and R₄ are defined above.
XX: 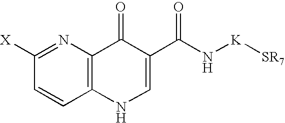
wherein X, K, and R₇ are defined above.
XXI: 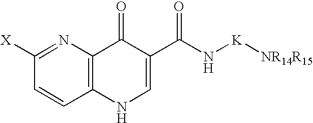
wherein X, K, R₁₄, and R₁₅ are defined above.
XXII: 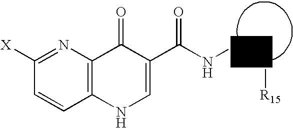
wherein X, K, and R₁₅ are defined above.
XXIII: 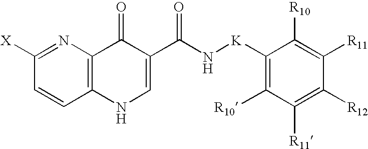
wherein R₁₀, R₁₇ are the same or different and may be selected from hydrogen, (C₁-C₆)alkyl, halogen, hydroxy, lower alkoxy having 1-6 carbon atoms, or cycloalkoxy having 3-7 carbon atoms;
R₁₁, R₁₁^{′}, and R₁₂ are the same or different and may be selected from hydrogen, (C₁-C₆)alkyl, halogen, hydroxy, —OR₄, —CR₇(R₉)NR₅R₆, —CR₇(R₉)OR₄; or
R₁₁ and R₁₂ taken together with the atoms to which they are attached form a (hetero)cyclic ring;
   and
R₉ is as defined above.
   XXIV: 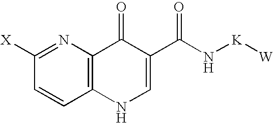
   wherein X and K are defined above; and W is heteroaryl.
   XXV: 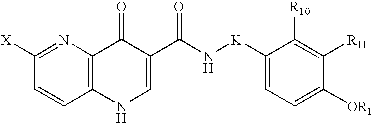
   wherein X, K, R₁, R₁₀, and R₁₁ are defined above.
   XXVI: 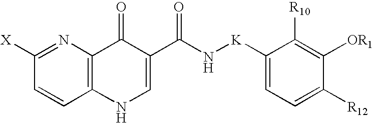
   wherein X, K, R₁, R₁₀, and R₁₂ are defined above.
   XXVII: 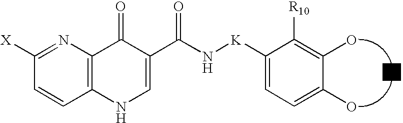
   wherein X, K, R₁₀, and G are defined above.
   XXVIII: 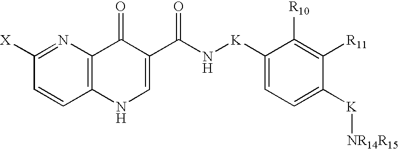
   wherein X, K, R₁₄, R₁₅, R₁₀, and R₁₁ are defined above.
   XXIX:

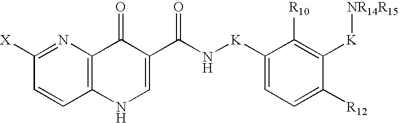

Other compounds prepared by the process of the present invention are encompassed by the following formulae:

XXX: 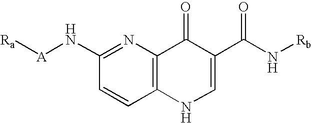
where A is C₁-C₆ alkylene;
Rₐ is phenyl optionally mono-, di-, or trisubstituted with halogen, lower alkyl, lower alkoxy, or mono- or di-C₁-C₆ alkylamino, or mono- or di-C₁-C₆ alkylamino lower alkyl; and
R_{b} is lower alkyl or lower cycloalkyl.

Compounds of Formula XXX made in accordance with the invention are those where A is methylene, Rₐ is phenyl optionally substituted with methyl or ethyl, and R_{b} is lower alkyl. Particular compounds of Formula XXX are those where A is methylene, Rₐ is phenyl and R_{b} is C₁-C₃ alkyl.

XXXI: 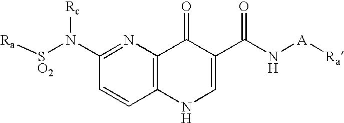
wherein A is C₁-C₆ alkylene;
Rₐ and Rₐ^{′} are independently phenyl groups optionally mono-, di-, or trisubstituted with halogen, lower alkyl, lower alkoxy, or mono- or di-C₁-C₆ alkylamino, or mono- or di-C₁-C₆ alkylamino lower alkyl; and
R_{c} is hydrogen or lower alkyl.

Other compounds of Formula XXXI made in accordance with the invention are those where A is methylene, Rₐ and Rₐ^{′} are independently phenyl optionally substituted with methyl or ethyl, and R_{c} is lower alkyl. Particular compounds of Formula XXXI are those where A is methylene, Rₐ is phenyl substituted in the para position with lower alkyl, Rₐ^{′} is phenyl, and R_{c} is C₁-C₃ alkyl.

XXXII: 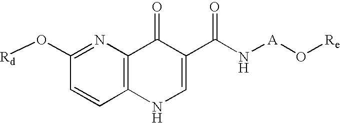
wherein A is C₁-C₆ alkylene;
R_{d} and Rₑ are independently lower alkyl groups.

Compounds of Formula XXXII made in accordance with the invention are those where A is C₂-C₄ alkylene. Particular compounds of Formula XXXII are those where A is C₂-C₄ alkylene,
R_{d} is C₁-C₃ alkyl, and Rₑ is C₂-C₄ alkyl.

XXXIII: 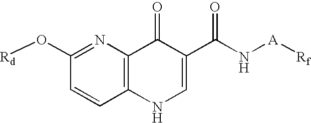
wherein A is C₁-C₆ alkylene;
R_{d} is lower alkyl; and
R_{f} is a group of the formula: 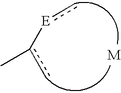
   where E is oxygen or nitrogen; and
M is C₁-C₃ alkylene or nitrogen.

Compounds of Formula XXXIII made in accordance with the invention are those where A is C₁-C₃ alkylene. Other compounds of Formula XXXIII are those where A is C₂-C₄ alkylene, R_{d} is C₁-C₃ alkyl, and R_{d} is C₂-C₄ alkyl. Other compounds of Formula XXXIII are those where A is C₂-C₄ alkylene, R_{d} is C₁-C₃ alkyl, Rₑ is C₂-C₄ alkyl, and E is nitrogen and M is methylene, E is oxygen and M is methylene or ethylene, or E and M are both nitrogen.

Still other compounds of Formula XXXIII made by the process of the invention are those where R_{f} is furanyl, tetrahydrofuranyl, or imidazolyl.

XXXIV: 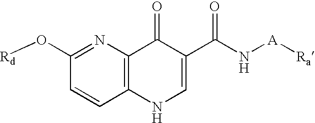
wherein A is C₁-C₆ alkylene; R_{d} is lower alkyl optionally substituted with amino or mono- or di(C₁-C₆)alkylamino; and Rₐ^{′} is phenyl optionally mono-, di-, or trisubstituted with halogen, lower alkyl, lower alkoxy, or mono- or di-C₁-C₆ alkylamino, or mono- or di-C₁-C₆ alkylamino lower alkyl.

Compounds of Formula XXXIV made in accordance with the invention are those where A is C₁-C₃ alkylene, Rₐ^{′} is phenyl optionally substituted with methyl or ethyl, and R_{d} is C₁-C₃ alkyl. Other compounds of Formula XXXIV are where A is methylene, Rₐ^{′} is phenyl optionally substituted with methyl or ethyl, and R_{d} is C₃-C₆ alkyl. Particular compounds of Formula XXXIV are sodium, potassium, or ammonium salts of the corresponding parent compound.

Other preferred compounds of Formula XXXIV made in accordance with the invention are those where Rₐ is phenyl substituted with mono- or di-(C₁-C₆) alkylamino lower alkyl.

XXXIVa: 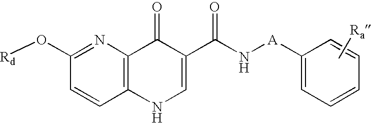
wherein A is C₁-C₆ alkylene;
R_{d} is lower alkyl; and Rₐ^{″} is phenyl, pyridyl, imidazolyl, pyrimidinyl, or pyrrolyl, each of which is optionally substituted with up to two groups selected from halogen, lower alkyl, lower alkoxy, mono- or di(C₁-C₆)alkylamino, or mono- or di-C₁-C₆ alkylamino lower alkyl.

Compounds of Formula XXXIVa made in accordance with the invention are those where Rₐ^{″} is imidazolyl and R_{d} is C₁-C₃ alkyl. Particular compounds of Formula XXXIVa made in accordance with the invention are those where A is methylene, R_{a″} is imidazolyl, and R_{d} is C₃-C₆ alkyl.

XXXV: 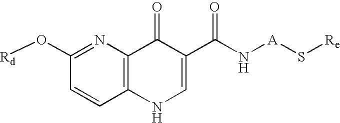
wherein A is C₁-C₆ alkylene; and
R_{d} and Rₑ are independently lower alkyl groups.

Compounds of Formula XXXV made in accordance with the invention are those where A is C₁-C₃ alkylene. Particular compounds of Formula XXXV made by the process of the invention are those where A is C₁-C₃ alkylene, R_{d} is C₁-C₃ alkyl, and Rₑ is C₁-C₃ alkyl.

XXXVI: 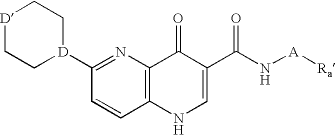
wherein D is nitrogen or CH;
D′ is nitrogen or oxygen;
A is C₁-C₆ alkylene; and Rₐ^{′} is phenyl, pyridyl, or thiazol, each of which is optionally mono-, di-, or trisubstituted with halogen, lower alkyl, lower alkoxy, or mono- or di-C₁-C₆ alkylamino, or mono- or di-C₁-C₆ alkylamino lower alkyl.

Compounds of Formula XXXVI made in accordance with the invention are those where A is C₁-C₃ alkylene, Rₐ^{′} is phenyl optionally substituted with lower alkyl or halogen, and D is nitrogen. Other compounds of Formula XXXVI made in accordance with the invention are where A is methylene, Rₐ^{′} is phenyl optionally substituted with lower alkyl or halogen, D is nitrogen, and D′ is oxygen.

XXXVII: 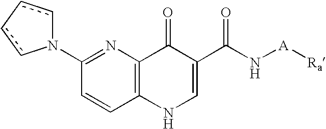
wherein A is C₁-C₆ alkylene; and
Rₐ^{′} is hydrogen; Rₐ^{′} is thienyl or phenyl, each of which is optionally mono-, di-, or trisubstituted with halogen, lower alkyl, lower alkoxy, or mono- or di-C₁-C₆ alkylamino, or mono- or di-C₁-C₆, alkylamino lower alkyl.

Compounds of Formula XXXVII made in accordance with the invention are those where A is C₁-C₃ alkylene, and Rₐ^{′} is phenyl optionally substituted with lower alkyd or halogen. Other compounds of Formula XXXVII made in accordance with the invention are those where A is methylene, Rₐ^{′} is phenyl optionally substituted with lower alkyl, lower alkoxy or halogen.

XXXVIII: 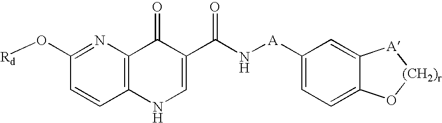
wherein A is C₁-C₆ alkylene; and
R_{d} is lower alkyl; A′ represents oxygen or methylene; and r is a n integer of from 1-3.

Specific compounds of Formula XXXVIII made in accordance with the invention are those where A is C₁-C₃ alkylene. Other compounds of Formula XXXVIII made in accordance with the invention are those where A is C₁-C₃ alkylene, and R_{d} is C₁-C₃ alkyl. 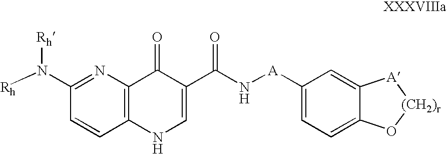
wherein A is C₁-C₆ alkylene; and
Rₕ and Rₕ^{′} are independently hydrogen or lower alkyl, where each alkyl is optionally substituted with lower alkoxy; A′ represents oxygen or methylene; and r is an integer of from 1-3.

Other compounds of Formula XXXVIIIa made in accordance with the invention are those where A is C₁-C₃ alkylene. Additional compounds of Formula XXXVIIIa made in accordance with the invention are those where A is C₁-C₃ alkylene, and Rₕ is C₁-C₃ alkyl.
XXXIX: 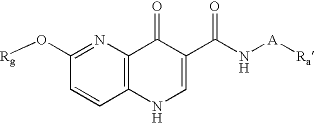
wherein A is C₁-C₆ alkylene; R_{g} is lower alkoxy lower alkyl; and Rₐ^{′} is phenyl optionally mono-, di-, or trisubstituted with halogen, lower alkyl, lower alkoxy, or mono- or di-C₁-C₆ alkylamino, or mono- or di-C₁-C₆ alkylamino lower alkyl.
XXXX: 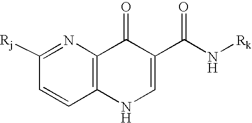
wherein Rⱼ is halogen or lower alkoxy; and Rₖ is lower alkyl or cycloalkyl each of which is optionally substituted with hydroxy, lower alkyl, or lower alkoxy; or Rₖ is phenyl (C₁-C₆) alkyl where the phenyl group is optionally mono-, di-, or trisubstituted with halogen, lower alkyl, lower alkoxy, or mono- or di-C₁-C₆ alkylamino, or mono- or di-C₁-C₆ alkylamino lower alkyl.
XXXXI: 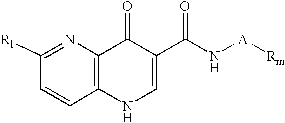
wherein A is C₁-C₆ alkylene; Rₗ lower alkoxy, benzyloxy, lower alkoxy lower alkoxy, amino, or mono- or di-(C₁-C₆)alkylamino; and Rₘ is pyranyl, dihydropyranyl, tetrahydropyranyl, or hexahydropyranyl, pyridine, dihydropyridine, tetrahydropyridine, or piperidine.

Compounds of Formula XXXXI made in accordance with the invention are those where Rₗ is lower alkoxy and Rₘ is tetrahydropyranyl.
XXXXII: 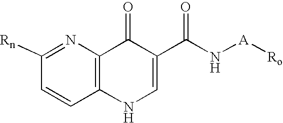
wherein A is C₁-C₆ alkylene; Rₙ is lower alkoxy, benzyl, or a group of the formula: 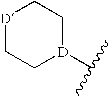
where D is nitrogen or CH; and D′ is nitrogen or oxygen; and Rₒ is pyranyl, 2-or 3-thienyl; or Rₒ is 2-, 4-, or 5-thiazolyl or 2-, 4-, or 5-imidazolyl, each of which may be optionally substituted with lower alkyl.
XXXXIII: 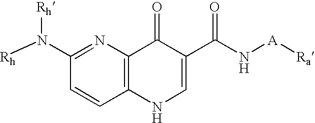
wherein A is C₁-C₆ alkylene; Rₕ and Rₕ′ are independently hydrogen or lower alkyl, where each lower alkyl is optionally substituted with lower alkoxy; and Rₐ^{′} is phenyl optionally mono-, di-, or trisubstituted with halogen, lower alkyl, lower alkoxy, or mono- or di-C₁-C₆ alkyl, amino, or mono- or di-C₁-C₆ alkylamino lower alkyl; or Rₐ^{′} is thienyl optionally substituted with lower alkyl.
XXXXIV: 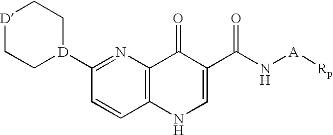
wherein A is C₁-C₆ alkylene; D is nitrogen or CH; D′ is nitrogen or oxygen; and Rₚ is lower alkyl or lower alkyl optionally substituted with lower alkoxy.
XXXXV: 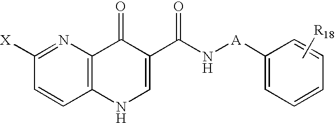
wherein A is C₁-C₆ alkylene; X is defined as above for Formula IV; and R₁₈ is:
(i) amino or mono- or di(C₁-C₆)alkylamino; or
(ii) lower alkyl optionally substituted with 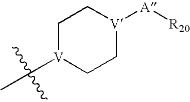
   where V and V′ are independently CH or nitrogen; A″ is C₁-C₆ alkylene; and R₂₀ is phenyl, pyridyl, or pyrimidinyl, each of which is optionally mono-, di-, or trisubstituted independently with halogen, hydroxy, C₁-C₆ alkoxy, amino, or mono- or di(C₁-C₆)alkylamino.

Compounds of Formula XXXXV made in accordance with the invention are those where V is nitrogen and X is C₁-C₆ alkoxy or C₁-C₆ alkyl optionally substituted with up to three halogen atoms. Particular compounds of XXXXV made in accordance with the invention are those where V and V′ are nitrogen; X is C₁-C₃ alkoxy or C₁-C₃ alkyl optionally substituted with up to three halogen atoms; A″ is methylene or ethylene; and R₂₀ is halogenated phenyl. In a specific embodiment, R₂₀ group is 4-fluorophenyl. Other compounds of XXXXV made in accordance with the invention are those where X is 2,2,2-trifluoroethyl; V and V′ are nitrogen; R₂₀ is halogenated phenyl; and A and A″ are methylene or ethylene.

In certain situations, compounds of Formula IV made in accordance with the invention may contain one or more asymmetric carbon atoms, so that the compounds can exist in different stereoisomeric forms. These compounds can be, for example, racemates or optically active forms. In these situations, the single enantiomers, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral HPLC column.

Representative compounds encompassed by Formula IV may be prepared by the process of the present invention include, but are not limited to the compounds in Table I and their pharmaceutically acceptable acid and base addition salts. In addition, if the compound of the invention is obtained as an acid addition salt, the free base can be obtained by basifying a solution of the acid salt. Conversely, if the product made in accordance with the invention is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds.

Non-toxic pharmaceutical salts of compounds made in accordance with the invention include such salts of acids as hydrochloric acid, phosphoric acid, hydrobromic acid, sulfuric acid, sulfinic acid, formic acid, toluenesulfonic acid, methanesulfonic acid, nitric acid, benzoic acid, citric acid, tartaric acid, maleic acid, hydroiodic acid, alkanoic acids such as acetic acid, HOOC—(CH₂)ₙ—ACOOH where n is 0-4, and the like. Non-toxic pharmaceutical base addition salts of compounds made in accordance with the invention include salts of bases such as sodium, potassium, calcium, ammonium, and the like. Those skilled in the art will recognize a wide variety of non-toxic pharmaceutically acceptable addition salts.

The process of the present invention also encompasses the acylated prodrugs of the compounds of Formula IV. Those skilled in the art will recognize various synthetic methodologies which may be employed to prepare non-toxic pharmaceutically acceptable addition salts and acylated prodrugs of the compounds encompassed by Formula IV.

“Lower alkyl” as used in the present invention is to encompass straight or branched chain alkyl groups having 1-6 carbon atoms, such as, for example, methyl, ethyl, propyl, isopropyl, n-butyl, sec-butyl, tert-butyl, pentyl, 2-pentyl, isopentyl, neopentyl, hexyl, 2-hexyl, 3-hexyl, and 3-methylpentyl.

By “cycloalkyl” in the present invention is meant cycloalkyl groups having 3-7 atoms such as, for example cyclopropyl, cyclobutyl, cyclopentyl, cyclohexyl, and cycloheptyl.

By “aryl” is meant an aromatic carbocyclic group having a single ring (e.g., phenyl), multiple rings (e.g., biphenyl), or multiple condensed rings in which at least one is aromatic, (e.g., 1,2,3,4-tetrahydronaphthyl, naphthyl, anthryl, or phenanthryl), which is optionally mono-, di-, or trisubstituted with, e.g., halogen, lower alkyl, lower alkoxy, lower alkylthio, trifluoromethyl, lower acyloxy, aryl, heteroaryl, and hydroxy.

By “lower alkoxy” in the present invention is meant straight or branched chain alkoxy groups having 1-6 carbon atoms, such as, for example, methoxy, ethoxy, propoxy, isopropoxy, n-butoxy, sec-butoxy, tert-butoxy, pentoxy, 2-pentyl, isopentoxy, neopentoxy, hexoxy, 2-hexoxy, 3-hexoxy, and 3-methylpentoxy.

By “cycloalkoxy” in the present invention is meant cycloalkylalkoxy groups having 3-7 carbon atoms where cycloalkyl is defined above.

By “halogen” in the present invention is meant fluorine, bromine, chlorine, and iodine.

By “heteroaryl” (aromatic heterocycle) in the present invention is meant one or more aromatic ring systems of 5-, 6-, or 7-membered rings containing at least one and up to four hetero atoms selected from nitrogen, oxygen, or sulfur. Such heteroaryl groups include, for example, thienyl, furanyl, thiazolyl, imidazolyl, (is)oxazolyl, pyridyl, pyrimidinyl, (iso)quinolinyl, naphthyridinyl, benzimidazolyl, and benzoxazolyl.

Specific examples of heteroaryl groups are the following: 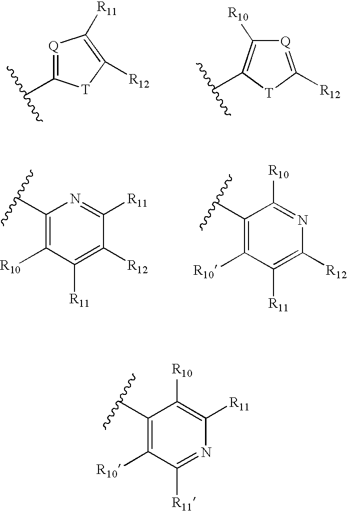
wherein Q is nitrogen or —CR₉; T is —NR₇, oxygen, or sulfur; and R₉, R₁₀, R₁₀′, R₁₁, R₁₁′, R₁₂ are as defined above.

In particular, the process of the invention permits forming compounds wherein Y represents a carbocyclic group, and is attached to the amide nitrogen by a single bond. The result is an amide of the formula: 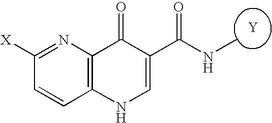
where X is defined as above and 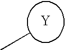
represents the Y carbocyclic group.

In accordance with the process of the invention, where X is a carbocyclic group, such moiety or group includes both aromatic heterocycles (heteroaryl), unsaturated heterocyclic ring systems, and saturated heterocyclic ring systems. Examples of such groups are imidazolyl, pyrrolidinyl, morpholinyl, piperazinyl, piperidinyl. Certain X carbocyclic groups are linked to the parent naphthyridine moiety by a nitrogen atom in the X carbocyclic group. Thus, for example, when pyrrolidinyl is the X carbocyclic group, it is preferably a 1-pyrrolidinyl group of the formula:

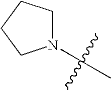

In accordance with the process of the invention, where Y is a carbocyclic group, such moiety or group includes both aromatic heterocycles (heteroaryl groups), unsaturated heterocyclic ring systems, and saturated heterocyclic ring systems. Examples of such groups are imidazolyl, pyrrolidinyl, morpholinyl, piperazinyl, or piperidinyl. Preferred Y carbocyclic groups are linked to the parent naphthyridine carboxamide group by a nitrogen atom in the Y carbocyclic group. Thus, for example, when piperidinyl is the Y carbocyclic group, it is favorably a 1-piperidinyl group of the formula:

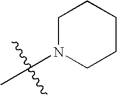

By “optionally substituted phenyl” as used herein is meant phenyl groups that are unsubstituted or substituted with up to 3 groups selected independently from halogen, hydroxy, lower alkyl, lower alkoxy, trifluoromethyl, and mono- or di-lower alkylamino.

Representative compounds that may be prepared by the process of the present invention are shown below in Table 1.

TABLE 1

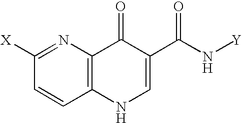

| | X | Y |
|---|---|---|
| 1. | C₆H₅CH₂NH— | —CH₂CH₂CH₂CH₃ |
| 2. | p-CH₃C₆H₄SO₂N(CH₃)— | —CH₂C₆H₅ |
| 3. | CH₃CH₂O— | —CH₂CH₂CH₂OCH(CH₃)2 |
| 4. | CH₃CH₂O— | 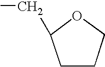 |
| 5. | CH₃O— | —CH₂CH₂SCH₂CH₃ |
| 6. | CH₃CH₂O— | 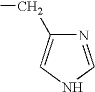 |
| 7. | O(CH₂CH₂)₂N— | —CH₂C₆H₄F-o |
| 8. | (CH₂CH₂CH₂CH₂)N— | —CH₂C₆H₄OCH₃-p |
| 9. | CH₃CH₂O— | 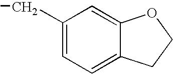 |
| 10. | CH₃CH₂O— | —CH₂C₆H₄CH₂NHCH₃-p |
| 11. | CH₃CH₂O— | —CH₂C₆H₅ |

### DETAILED DESCRIPTION OF THE INVENTION

The process of the present invention and the preparation of the compounds of the present invention are illustrated in Scheme 1. The preparation of the compounds of formulas II-IV are summarized in Scheme 1, wherein, unless otherwise indicated, X and Y are as defined above.

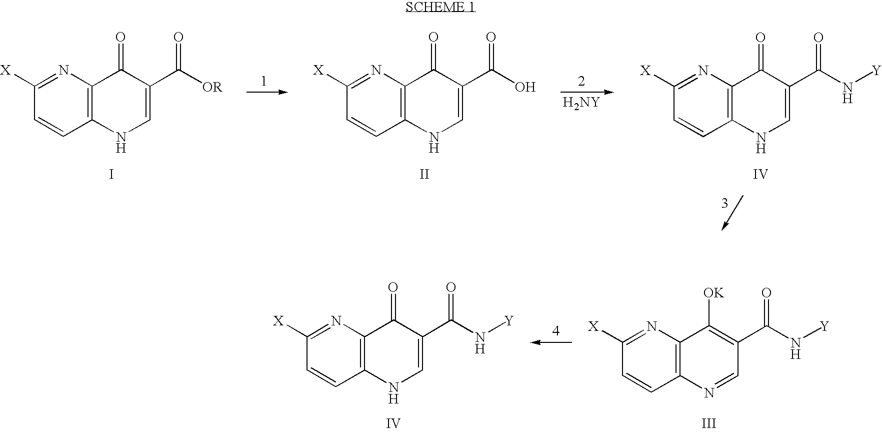

Overall, the synthetic sequence of the scheme involves saponification of the ester having structure I and acidification to form the free carboxylic acid having structure II (step 1), treatment of compound II with a primary amine and a coupling agent, preferably 1,1-carbonyldiimidazole to form an amide having structure IV (step 2) and, optionally, treatment of the amide of structure IV with a strong base such as potassium t-butoxide to form a salt having structure III (step 3), purification of the salt of structure III and then acidification of the salt of structure III to form the amide of structure IV (step 4).

In step 1 of Scheme 1 the ester having structure I is treated with an excess of strong base such as NaOH, KOH, CeOH, LiOH or other hydroxide or alkoxide base such as KOC(CH₃)₃, preferably NaOH or KOH in an aqueous alcoholic solvent such as methanol, ethanol or isopropanol, preferably ethanol and heated at reflux for about 1 hour to about 12 hours, with about 1 hour preferred. The solution is cooled to about 5° C. to about 25° C., with about 22° C. preferred. The solution is then gradually acidified with a strong mineral acid, preferably concentrated HCl, preferably to about pH 1 to about pH 4, most preferably, about pH 2 over a period of about 1 to about 2 hours with the final temperature being about 7° C. to about 11° C. To facilitate adjusting the pH to the preferred range, bases such KOH, NaOH, LiOH, CeOH, alkali metal alkoxides, phosphate bases, bicarbonate bases or carbonate bases may be used, with NaOH and KOH preferred. The resultant slurry is stirred for about 1 to about 5 hours at about 12° C. to about 16° C. and is then filtered, washed with water and dried under vacuum. In a preferred variation, in order to facilitate filtration, the acidified slurry is aged at elevated temperature prior to filtration, typically by heating to reflux with stirring for about 30 minutes to about 16 hours, more preferably about 1 hour to about 2 hours and is then cooled to about room temperature and filtered. The resultant product may be optionally recrystallized.

In step 2 of Scheme 1 compound II is treated with a primary amine and a coupling agent, such as thionyl chloride, oxalyl chloride or 1,1-carbonyldiimidazole (CDI) with CDI preferred, in a solvent, preferably an amide solvent such as dimethylformamide (DMF), dimethylacetamide (DMAC) or N-methylpyrrolidone (NMP), with DMF most preferred, to form an amide having structure IV. In an alternate, but less preferred route a mixed anhydride is formed by treating Compound II with an acid halide or an alkyl halo formate and a tertiary amine, preferably a trialkyl amine, to form a mixed anhydride which is treated with the primary amine to form amide IV. Amide IV may be optionally recrystallized from a solvent such as DMF or DMAC.

In step 3, the amide of structure IV is treated with a strong base, preferably a strong base such as potassium t-butoxide, by heating in a solvent, preferably an ethereal solvent containing about 3% to about 10% water, with solvents such as dioxane or tetrahydrofuran (THF) preferred, and THF most preferred, to form a salt, preferably a potassium salt, having formula III of Scheme I. The potassium salt formed in this way can be readily filtered and recrystallized from THF. The corresponding Na, Li, or Ce salts, which are less preferred, may be formed using the corresponding strong base.

In step 4, the salt corresponding to compound III, preferably the potassium salt, is slurried in water and acidified with a strong acid to a pH of about 2 to a pH of about 3.5, preferably with a mineral acid, most preferably with HCl, to form compound IV. Compound IV is readily filtered, washed with water and dried to yield highly purified compound IV.

The present invention is illustrated by the following examples, but it is not limited to the details thereof.

### EXAMPLE 1

### 6-Ethoxy-4-oxo-1,4-dihydro-1,5-naphthyridine-3-carboxylic acid

To a clean 1000 gallon glass lined reactor was charged 185 Kg of ethyl 6-ethoxy-4-oxo-1,4-dihydro-[1,5]-naphthyridine-3-carboxylic acid, ethyl ester, 1480 L water and 437 Kg of 2-B EtOH. To the slurry was added 150 Kg of 50% NaOH solution and 185 L water at temperatures below 40° C. The reaction was heated to reflux and held for 2 hrs. The solution was cooled to 45° C. and transferred through a preheated polish filter to a second clean 1000 gallon glass lined reactor. The empty reactor and polish filter were rinsed with 110 L water. At 25° C., the reaction filtrate was adjusted to a pH of 2-4 with filtered concentrated HCl and 50% NaOH. The slurry was heated to reflux and held for 1 hour. The slurry was allowed to stir for 1 hour at 22° C. and then filtered on a 240 L centrifuge in 4 loads. Each load was washed with 95 L filtered water and together dried in a vacuum tray dryer at 66° C. until the KF was 0.2%. This procedure afforded a 94% yield (155 Kg) of spec-free product. mp 260-268° C. Anal. Calc. for C₁₁H₁₀N₂O₄: C 56.41; H, 4.30; N, 11.96. Found: C, 56.30; H, 3.96; N, 11.93. ¹H NMR (400 MHz, DMSO) 8.85 (s, 1H), 8.12 (d, J=9.2, 1H), 7.33 (d, J=9.2, 1H), 4.44 (q, J=7.2, 2H), 1.38 (t, J=7.2, 3H). ¹³C NMR (125 MHz, DMSO) □ 176.6, 166.5, 160.8, 142.8, 137.2, 133.1, 132.0, 118.9, 110.7, 62.1, 14.2.

### EXAMPLE 2

### N-benzyl-6-ethoxy-4-oxo-1,4-dihydro-1,5-naphthyridine-3-carboxamide

A mixture of 6-ethoxy-4-oxo-1,4-dihydro-1,5-naphthyridine-3-carboxylic acid (29 kg, 123.82 M) and 1,1-carbonyldiimidazole (22 kg, 135.64 M) in 31 gallons of DMF was heated to 90° C. under nitrogen for 2 hours during which time there was gas evolution. The mixture was cooled to 35° C. and benzyl amine (14.6 kg, 136.24 M) was added and the mixture allowed to stir at 35° C. for 2 hours, after which time the reaction was essentially complete. The mixture was then diluted with 192 gallons of water. The resultant slurry was allowed to granulate for 4 hours and then the product was isolated via filtration. The filtered product was then dried 24 hours at 45° C. to essentially constant weight. The product, N-benzyl-6-ethoxy-4-oxo-1,4-dihydro-1,5-naphthyridine-3-carboxamide (38.9 kg, 97% yield) had NMR and HPLC consistent with a known sample. mp 199° C. Anal. Calc. for C₁₈H₁₇N₃O₃: C, 66.86; H, 5.30; N, 13.00. Found: C, 66.69; H, 5.18; N, 12.91. ¹H NMR (500 MHz, DMSO) □12.73 (br s, 1H), 10.61 (br t, J=5.6, 1H), 8.73 (s, 1H), 8.05 (d, J=9.0, 1H), 7.35 (d, J=4.4, 2H), 7.27 (dd, J=4.4, 8.6, 2H), 7.22 (d, J=9.0, 1H), 4.56 (d, J=5.8, 2H), 4.42 (q, J=7.0, 2H), 1.35 (t, J=7.1, 3H). ¹³C NMR (125 MHz, DMSO) 174.5, 164.5, 160.1, 141.6, 139.3, 138.6, 132.1, 131.5, 128.4, 127.3, 126.9, 117.5, 113.6, 61.7, 42.1, 14.3.

### EXAMPLE 3

### N-benzyl-6-ethoxy-4-oxo-1,4-dihydro-1,5-naphthyridine-3-carboxamide potassium salt

A mixture of N-benzyl-6-ethoxy-4-oxo-1,4-dihydro-1,5-naphthyridine-3-carboxamide (38 kg, 117.52 M), 80 gallons of THF, potassium t-butoxide (13.5 kg, 120.54 M) and 19 l of water was stirred to form a slurry and heated at reflux for 1 hour. The resultant solution was then filtered at a temperature maintained just below reflux. An additional 10 gallons of THF heated to 55° C. was used to rinse the filtration equipment and added to the main filtrate. An additional 80 gallons of THF was added to the filtrate which was then concentrated at atmospheric pressure to 55 gallons. The mixture was allowed to cool and granulate for 16 hours. A first crop of 27.4 kg was obtained as a wet cake. The NMR/HPLC of the product was consistent with N-benzyl-6-ethoxy-4-oxo-1,4-dihydro-1,5-naphthyridine-3-carboxamide potassium salt. The mother liquor from the first crop was diluted with 55 gallons of THF, concentrated atmospherically to a final volume of 15 gallons, allowed to cool and granulate for 16 hours to yield a second crop (17.4 kg, wet cake). mp 208° C. ¹H NMR (400 MHz, DMSO) □8.72 (s, 1H), 8.03 (d, J=9.2, 1H), 7.34-7.23 (m, 5H), 7.21 (d, J=9.2, 1H), 4.54 (d, J=5.2, 2H), 4.39 (q, J=7.2, 2H), 1.33 (t, J=7.2, 3H). ¹³C NMR (100 MHz, DMSO) 172.8, 168.8, 159.2, 150.9, 143.2, 141.2, 141.1, 140.3, 129.0, 127.9, 127.2, 114.8, 112.8, 61.5, 42.4, 15.2.

### EXAMPLE 4

### N-benzyl-6-ethoxy-4-oxo-1,4-dihydro-1,5-naphthyridine-3-carboxamide-clinical purity

A mixture of N-benzyl-6-ethoxy-4-oxo-1,4-dihydro-1,5-naphthyridine-3-carboxamide potassium salt (27.4 kg, THF wet) and 120 gallons of UPS water was stirred at 19° C. to form a slurry. The slurry was acidified with 6 L of 37% HCl yielding a pH of 2.4. The slurry was allowed to granulate 4 hours at 20° C. The product was isolated via filtration and was then washed with two 10 gallon portions of 40° C. water. The product was dried in vacuum at 45-50° C. for 48 hours under a nitrogen bleed. The product, N-benzyl-6-ethoxy4-oxo-1,4-dihydro-1,5-naphthyridine-3-carboxamide (16.2 kg, 42% yield) had NMR/HPLC, ash and powder x-ray indicating clinical grade purity. mp 199° C. Anal. Calc. for C₁₈H₁₇N₃O₃: C, 66.86; H, 5.30; N, 13.00. Found: C, 66.69; H, 5.18; N, 12.91. ¹H NMR (500 MHz, DMSO) 12.73 (br s, 1H), 10.61 (br t, J=5.6, 1H), 8.73 (s, 1H), 8.05 (d, J=9.0, 1H), 7.35 (d, J=4.4, 2H), 7.27 (dd, J=4.4, 8.6, 2H), 7.22 (d, J=9.0, 1H), 4.56 (d, J=5.8, 2H), 4.42 (q, J=7.0, 2H), 1.35 (t, J=7.1, 3H). ¹³C NMR (125 MHz, DMSO) 174.5, 164.5, 160.1, 141.6, 139.3, 138.6, 132.1, 131.5, 128.4, 127.3, 126.9, 117.5, 113.6, 61.7, 42.1, 14.3.

A similar procedure was applied to the second crop of potassium salt obtained in Example 3, but a second water granulation with USP water was needed to reduce the ash levels. The final product, N-benzyl-6-ethoxy-4-oxo-1,4-dihydro-1,5-naphthyridine-3-carboxamide (9.4 kg, 24% yield) was identical with the material from the first crop and had NMR/HPLC, ash and powder x-ray indicating clinical grade purity.

1. A process of preparing a compound of the formula IV: 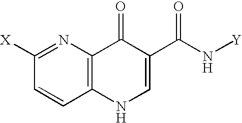
wherein X is hydrogen, halogen, —OR₁, C₁-C₆ alkyl optionally substituted with up to three groups selected independently from halogen and hydroxy, or —NR₂R₃; phenyl, naphthyl, 1-(5,6,7,8-tetrahydro)naphthyl or 4-(1,2-dihydro)indenyl, pyridinyl, pyrimidyl, isoquinolinyl, 1,2,3,4-tetrahydroisoquinolinyl, benzofuranyl, benzothienyl, each of which is optionally substituted with up to three groups selected from halogen, C₁-C₆ alkyl, C₁-C₄ alkoxy, Cl-C₆ alkylthio, hydroxy, amino, mono or di(C₁-C₆)alkylamino, cyano, nitro, trifluoromethyl or trifluoromethoxy; or, a carbocyclic group containing from 3-7 members, up to two of which members are optionally hetero atoms selected from oxygen and nitrogen, where the X carbocyclic group is optionally substituted with one or more groups selected from halogen, alkoxy, mono- or dialkylamino, sulfonamide, azacycloalkyl, cycloalkylthio, alkylthio, phenylthio, or a heterocyclic group;
Y is lower alkyl having 1-8 carbon atoms optionally substituted with up to two groups selected from halogen, alkoxy, mono- or dialkylamino, sulfonamide, azacycloalkyl, cycloalkylthio, alkylthio, phenylthio, a heterocyclic group, —OR₄, —NR₅R₆, SR₇, or aryl; or a carbocyclic group having from 3-7 members atoms, where up to three of which members are optionally hetero atoms selected from oxygen and nitrogen and where any member of the Y carbocyclic group is optionally substituted with halogen, —OR₄, —NR₅R₆, SR₇, aryl or a heterocyclic group;
R₁ and R₄ are independently hydrogen, lower alkyl having 1-6 carbon atoms, or cycloalkyl having 3-7 carbon atoms, where each alkyl may be optionally substituted with —OR₄, or —NR₅R₆;
R₂ and R₃ are independently hydrogen, lower alkyl optionally mono- or disubstituted with alkoxy, aryl, halogen, or mono- or di-lower alkyl; aryl or aryl(C₁-C₆)alkyl where each aryl is optionally substituted with up to three groups selected from halogen, hydroxy, C₁-C₆ alkyl, C₁-C₆ alkoxy, or mono- or di (C₁-C₆) alkylamino; cycloalkyl having 3-7 carbon atoms optionally mono or disubstituted with halogen, alkoxy, or mono- or di-lower alkyl; or —SO₂R₈;
R₅ and R₆ have the same definitions as R₂ and R₃, respectively;
R₇ is hydrogen, lower alkyl having 1-6 carbon atoms, or cycloalkyl having 3-7 carbon atoms; and
R₈ is lower alkyl having 1-6 carbon atoms, cycloalkyl having 3-7 carbon atoms, or optionally substituted phenyl;
said process comprising the step of treating a compound of the formula III: 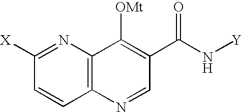
wherein Mt is K, Na, Ce, Li or quaternary (C₁-C₆)alkyl ammonium with a strong acid.

2. The process of claim 1 wherein the compound of formula III is (1) prepared by treating a compound of the formula IV: 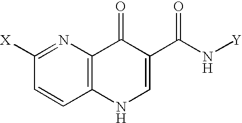
with a strong base, MtOBs, wherein Mt is K, Na, Ce, Li or quaternary alkyl ammonium and OBs is hydroxide or (C₁-C₆)alkoxide in a solvent and (2) purified.

3. The process of claim 2 wherein the compound of formula IV is prepared by treating a compound of the formula II: 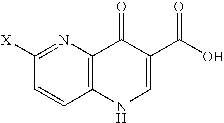
with a primary amine YNH₂ and 1,1-carbonyldiimidazole.

4. The process of claim 3 wherein the compound of formula II is prepared by treating a compound of the formula I: 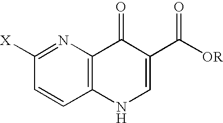
wherein R is (C₁-C₆)alkyl, with (1) a strong base and (2) an acid.

5. A process of preparing a compound of the formula IVA: 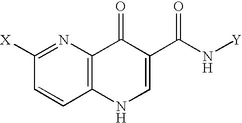
wherein X is:
(i) hydrogen, halogen, mono- or dialkylamino, alkoxy;
(ii) a group of the formula: 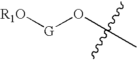
where G is lower alkylene having 1-6 carbon atoms, or a cyclic group of the formula 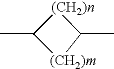
where n is 0, 1, or 2, and m is an integer of from 1 t o 5, with the proviso that the sum of n+m is not less than 1 or greater than 5; and R₁ is hydrogen, lower alkyl, or (C₃-C₇)cycloalkyl, where the alkyl or cycloalkyl is optionally substituted with halogen, lower alkoxy, or mono- or di(C₁-C₆)alkylamino;
(iii) a group of the formula: 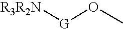
where G is as defined above for ii; and R₂ an d R₃ independently represent hydrogen, lower alkyl having 1-6 carbon atoms, cycloalkyl having 3-7 carbon atoms, —SO₂ R₈ where R₈ is (C₁-C₆)alkyl, (C₃-C₇)cycloalkyl, or optionally substituted phenyl, or R₂ and R₃ together with the nitrogen atom to which they are attached form a heterocyclic moiety selected from imidazolyl, pyrrolidinyl, morpholinyl, piperazinyl, or piperidinyl;
(iv) a group of the formula: 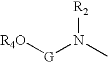
where R₂ is as defined above for iii;
R₄ is hydrogen, lower alkyl having 1-6 carbon atoms, or cycloalkyl having 3-7 carbon atoms, and may be optionally substituted with one or more (C₁-C₆)alkoxy or mono- or di(C₁-C₆)alkylamino groups; and
G is as defined above for ii;
(v) a group of the formula: 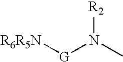
where R₂ and G are as defined above for iv and ii, respectively, and R₅ and R₆ independently represent hydrogen, lower alkyl having 1-6 carbon atoms, cycloalkyl having 3-7 carbon atoms, —SO₂ R₈ where R₈ is (C₁-C₆)alkyl, (C₃-C₇)cycloalkyl, or optionally substituted phenyl, or R₅ and R₆ together with the nitrogen atom to which they are attached form a heterocyclic moiety;
(vi) a group of the formula: 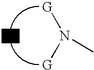
where G is as defined above for ii; or
(vii) a group of the formula: 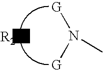
where each G is as defined above for ii; and
Y is
(viii) lower alkyl having 1-8 carbon atoms or cycloalkyl having 3-7 carbon atoms, any of which may be optionally substituted with one or more hydroxy, halogen, (C₁-C₆)alkoxy, alkoxyalkoxy where each alkoxy is (C₁-C₆)alkoxy, (C₁-C₆) alkylthio, (C₃-C₇)cycloalkylthio, aryl, heteroaryl, or mono- or di(C₁-C₆)alkylamino groups;
(ix) a group of the formula: 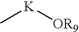
where K is lower alkylene having 1-6 carbon atoms optionally substituted with (C₁-C₆)alkyl or alkylene, or a cyclic group of the formula 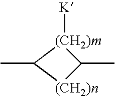
where K′ independently represents hydrogen or (C₁-C₆) alkyl or alkylene, n is 0, 1, or 2, and m is an integer of from 1 to 5, with the proviso that the sum of n+m is not less than 1 or greater than 5; and
R₉ is hydrogen, lower alkyl, or (C₃-C₇)cycloalkyl, where the alkyl or cycloalkyl is optionally substituted with halogen, lower alkoxy, or mono- or dialkylamino;
(x) a group of the formula: 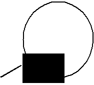
where K is defined as above in ix;
(xi) a group of the formula: 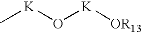
where
K is as defined above for ix, and
R₁₃ is hydrogen, lower alkyl having 1-6 carbon atoms, or cycloalkyl having 3-7 carbon atoms, where the alkyl and cycloalkyl groups are optionally substituted with one or more (C₁-C₆)alkoxy or mono- or di(C₁-C₆)alkylamino groups; and
(xii) a group of the formula: 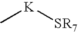
where K is as defined above for ix, and
R₇ is hydrogen, lower alkyl having 1-6 carbon atoms, or cycloalkyl having 3-7 carbon atoms; and
(xiii) a group of the formula: 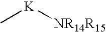
where K is as defined above for ix; and
R₁₄ and R₁₅ independently represent hydrogen, lower alkyl having 1-6 carbon atoms, cycloalkyl having 3-7 carbon atoms, —SO₂ R₈ where R₈ is as defined above, or R₁₄ and R₁₃ together with the nitrogen atom to which they are attached form a heterocyclic moiety;
(xiv) a group of the formula: 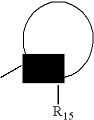
where K and R₁₅ are as defined above in ix and xii, respectively;
(xv) a group of the formula: 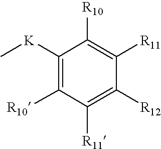
where K is as defined above for ix;
R₁₀ and R_{10′} are the same or different and are selected from hydrogen, (C₁-C₆)alkyl, halogen, hydroxy, lower alkoxy having 1-6 carbon atoms, or cycloalkoxy having 3-7 carbon atoms;
R₁₁, R_{11′}, and R₁₂ are the same or different and are selected from hydrogen, C₁-C₆ alkyl, halogen, hydroxy, —OR₄, —CR₇ (R₉)NR₅ R₆, —CR₉(R₁₆) OR₄, or R₁₁ and R₁₂ taken together with the atoms to which they are attached form a (hetero)cyclic ring; and
R₁₆ is hydrogen, lower alkyl having 1-6 carbon atoms, or cycloalkyl having 3-7 carbon atoms;
(xvi) a group of the formula: 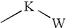
where K is as defined above for ix; and W is heteroaryl;
(xvii) a group of the formula: 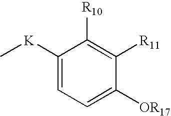
where K is as defined above for ix; R₁₀ and R₁₁ are as defined above for xv, and R₁₇ is hydrogen, lower alkyl, or (C_{.3}-C₇)cycloalkyl, where the alkyl or cycloalkyl is optionally substituted with halogen, lower alkoxy, or mono- or di(C₁-C₆)alkylamino;
(xviii) a group of the formula: 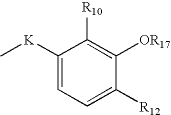
where K, R₁₀, R₁₂, and R₁₇ are as defined above;
(xix) a group of the formula: 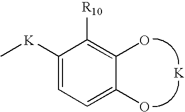
where each K is independently as defined above for ix and R₁₀ is defined above;
(xx) a group of the formula: 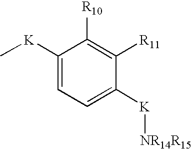
where K, R₁₀, R₁₁, R₁₄, and R₁₅ are as defined above;
(xxi) a group of the formula: 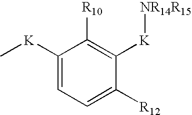
where K, R₁₀, R₁₂, R₁₄, and R₁₅ are as defined above;
(xxii) pyrimidinyl(C₁-C₆)alkyl or pyridyl(C₁-C₆)alkyl; or
(xxiii) a group of the formula: 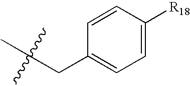
where R₁₈ represents hydrogen, amino, mono-, or di(C₁-C₆)alkylamino, or C₁-C₆ alkyl optionally substituted with a R₁₉ where R₁₉ represents: 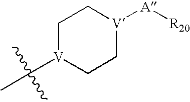
where V and V′ are independently CH or nitrogen; A″ is C₁-C₆ alkylene; and R₂₀ is phenyl, pyridyl, or pyrimidinyl, each of which is optionally mono-, di-, or trisubstituted independently with halogen, hydroxy, C₁-C₆ alkoxy, amino, or mono- or di(C₁-C₆)alkylamino; which comprises treating a compound of the formula IIIA: 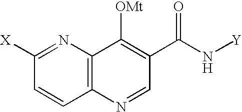
wherein Mt is K, Na, Ce, Li or quaternary (C₁-C₆)alkyl ammonium, with a strong acid.

6. The process of claim 5 wherein the compound of formula IIIA is (1) prepared by treating a compound of the formula IVA: 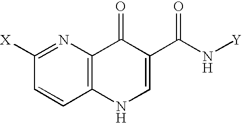
with a strong base MtOBs, wherein Mt is K, Na, Ce, Li or quaternary alkyl ammonium and OBs is hydroxide or (C₁-C₆)alkoxide and (2) purified.

7. The process of claim 6 wherein the compound of formula IVA is prepared by treating a compound of the formula IIA: 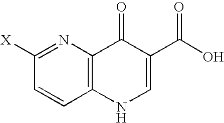
with a primary amine YNH₂ and 1,1 -carbonyldiimidazole.

8. The process of claim 7 wherein the compound of formula IIA is prepared by treating a compound of the formula IA: 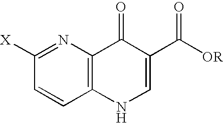
wherein R is C₁-C₆ alkyl, with (1) a strong base and (2) an acid.

9. The process according to claim 1 wherein X is ethoxy; Y is benzyl; and Mt is K.

10. The process according to claim 2 wherein X is ethoxy; Y is benzyl; Mt is K; MtOBs is KOH or potassium tertiary-butoxide; the solvent is aqueous tetrahydrofuran; and, the compound of formula III is purified by filtration and recrystallization.

11. The process according to claim 3 wherein X is ethoxy; Y is benzyl; and the primary amine is benzylamine.

12. The process according to claim 4 wherein X is ethoxy and R is ethyl.

13. The process according to claim 2 wherein the compound of formula III is purified by filtering a solution of the compound of formula III in the solvent, or recrystallizing the compound of formula III, or a combination thereof.

14. The process according to claim 6 wherein the compound of formula IIIA is purified by filtering a solution of the compound of formula IIIA in the solvent, or recrystallizing the compound of formula IIIA, or a combination thereof.

15. A process of preparing a compound of the formula IV: 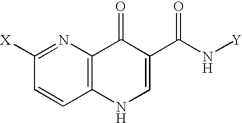
wherein X is hydrogen, halogen, —OR₁, C₁-C₆ alkyl optionally substituted with up to three groups selected independently from halogen and hydroxy, or —NR₂R₃; phenyl, naphthyl, 1-(5,6,7,8-tetrahydro)naphthyl or 4-(1,2-dihydro)indenyl, pyridinyl, pyrimidyl, isoquinolinyl, 1,2,3,4-tetrahydroisoquinolinyl, benzofuranyl, benzothienyl, each of which is optionally substituted with up to three groups selected from halogen, C₁-C₆ alkyl, C₁-C₄ alkoxy, C₁-C₆ alkylthio, hydroxy, amino, mono or di(C₁-C₆)alkylamino, cyano, nitro, trifluoromethyl or trifluoromethoxy; or, a carbocyclic group containing from 3-7 members, up to two of which members are optionally hetero atoms selected from oxygen and nitrogen, where the X carbocyclic group is optionally substituted with one or more groups selected from halogen, alkoxy, mono- or dialkylamino, sulfonamide, azacycloalkyl, cycloalkylthio, alkylthio, phenylthio, or a heterocyclic group;
Y is lower alkyl having 1-8 carbon atoms optionally substituted with up to two groups selected from halogen, alkoxy, mono- or dialkylamino, sulfonamide, azacycloalkyl, cycloalkylthio, alkylthio, phenylthio, a heterocyclic group, —OR₄, —NR₅R₆, SR₇, or aryl; or a carbocyclic group having from 3-7 members atoms, where up to three of which members are optionally hetero atoms selected from oxygen and nitrogen and where any member of the Y carbocyclic group is optionally substituted with halogen, —OR₄, —NR₅R₆, SR₇, aryl or a heterocyclic group;
R₁ and R₄ are independently hydrogen, lower alkyl having 1-6 carbon atoms, or cycloalkyl having 3-7 carbon atoms, where each alkyl may be optionally substituted with —OR₄, or —NR₅R₆;
R₂ and R₃ are independently hydrogen, lower alkyl optionally mono- or disubstituted with alkoxy, aryl, halogen, or mono- or di-lower alkyl; aryl or aryl(C₁-C₆)alkyl where each aryl is optionally substituted with up to three groups selected from halogen, hydroxy, C₁-C₆ alkyl, C₁-C₆ alkoxy, or mono- or di (C₁-C₆) alkylamino; cycloalkyl having 3-7 carbon atoms optionally mono or disubstituted with halogen, alkoxy, or mono- or di-lower alkyl; or —SO₂R₈;
R₅ and R₆ have the same definitions as R₂ and R₃, respectively;
R₇ is hydrogen, lower alkyl having 1-6 carbon atoms, or cycloalkyl having 3-7 carbon atoms; and
R₈ is lower alkyl having 1-6 carbon atoms, cycloalkyl having 3-7 carbon atoms, or optionally substituted phenyl;
said process comprising the step of treating a compound of the formula II: 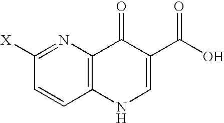
with a primary amine YNH₂ and a 1,1-carbonyldiimidazole.

16. The process of claim 15 wherein the compound of formula II is prepared by treating a compound of the formula I: 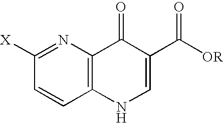
wherein R is (C₁-C₆)alkyl, with (1) a strong base and (2) an acid.

17. A process of preparing a compound of the formula IVA: 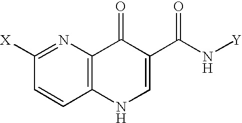
which comprises treating a compound of the formula IIA: 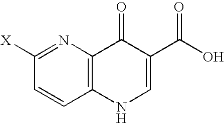
with a primary amine YNH₂ and a 1,1-carbonyldiimidazole.

18. The process of claim 17 wherein the compound of formula IIA is prepared by treating a compound of the formula IA: 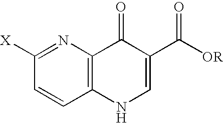
wherein R is (C₁-C₆)alkyl, with (1) a strong base and (2) an acid.
